SEQUENCE LISTING (12 sequences)

SEQ ID NO: 1 - protein, 297 aa, Artificial Sequence
  Wild-type murine CDK1 is mutated at position 32 (M32V) and position 80 (F80G)
MEDYIKIEKIGEGTYGVVYKGRHRVTGQIVAVKKIRLESEEEGVPSTAIREISLLKELRHPNIVSLQDVLMQDSRLYLIGEFLSMDLKKYLDSIPPGQFMDSSLVKSYLHQILQGIVFCHSRRVLHRDLKPQNLLIDDKGTIKLADFGLARAFGIPIRVYTHEVVTLWYRSPEVLLGSARYSTPVDIWSIGTIFAELATKKPLFHGDSEIDQLFRIFRALGTPNNEVWPEVESLQDYKNTFPKWKPGSLASHVKNLDENGLDLLSKMLVYDPAKRISGKMALKHPYFDDLDNQIKKM

SEQ ID NO: 2 - protein, 297 aa, Artificial Sequence
  Wild-type murine CDK1 is mutated at position 32 (M32V) and position 80 (F80A)
MEDYIKIEKIGEGTYGVVYKGRHRVTGQIVAVKKIRLESEEEGVPSTAIREISLLKELRHPNIVSLQDVLMQDSRLYLIAEFLSMDLKKYLDSIPPGQFMDSSLVKSYLHQILQGIVFCHSRRVLHRDLKPQNLLIDDKGTIKLADFGLARAFGIPIRVYTHEVVTLWYRSPEVLLGSARYSTPVDIWSIGTIFAELATKKPLFHGDSEIDQLFRIFRALGTPNNEVWPEVESLQDYKNTFPKWKPGSLASHVKNLDENGLDLLSKMLVYDPAKRISGKMALKHPYFDDLDNQIKKM

SEQ ID NO: 3 - protein, 297 aa, Artificial Sequence
  Wild-type murine CDK1 is mutated at position 75 (R75E) and position 80 (F80G)
MEDYIKIEKIGEGTYGVVYKGRHRVTGQIVAMKKIRLESEEEGVPSTAIREISLLKELRHPNIVSLQDVLMQDSELYLIGEFLSMDLKKYLDSIPPGQFMDSSLVKSYLHQILQGIVFCHSRRVLHRDLKPQNLLIDDKGTIKLADFGLARAFGIPIRVYTHEVVTLWYRSPEVLLGSARYSTPVDIWSIGTIFAELATKKPLFHGDSEIDQLFRIFRALGTPNNEVWPEVESLQDYKNTFPKWKPGSLASHVKNLDENGLDLLSKMLVYDPAKRISGKMALKHPYFDDLDNQIKKM

SEQ ID NO: 4 - protein, 297 aa, Artificial Sequence
  Wild-type murine CDK1 is mutated at position 75 (R75E) and position 80 (F80A)
MEDYIKIEKIGEGTYGVVYKGRHRVTGQIVAMKKIRLESEEEGVPSTAIREISLLKELRHPNIVSLQDVLMQDSELYLIAEFLSMDLKKYLDSIPPGQFMDSSLVKSYLHQILQGIVFCHSRRVLHRDLKPQNLLIDDKGTIKLADFGLARAFGIPIRVYTHEVVTLWYRSPEVLLGSARYSTPVDIWSIGTIFAELATKKPLFHGDSEIDQLFRIFRALGTPNNEVWPEVESLQDYKNTFPKWKPGSLASHVKNLDENGLDLLSKMLVYDPAKRISGKMALKHPYFDDLDNQIKKM

SEQ ID NO: 5 - protein, 297 aa, Artificial Sequence
  Wild-type human CDK1 is mutated at position 32 (M32V) and position 80 (F80G)
MEDYTKIEKIGEGTYGVVYKGRHKTTGQVVAVKKIRLESEEEGVPSTAIREISLLKELRHPNIVSLQDVLMQDSRLYLIGEFLSMDLKKYLDSIPPGQYMDSSLVKSYLYQILQGIVFCHSRRVLHRDLKPQNLLIDDKGTIKLADFGLARAFGIPIRVYTHEVVTLWYRSPEVLLGSARYSTPVDIWSIGTIFAELATKKPLFHGDSEIDQLFRIFRALGTPNNEVWPEVESLQDYKNTFPKWKPGSLASHVKNLDENGLDLLSKMLIYDPAKRISGKMALNHPYFNDLDNQIKKM

SEQ ID NO: 6 - protein, 297 aa, Artificial Sequence
  Wild-type human CDK1 is mutated at position 32 (M32V) and position 80 (F80A)
MEDYTKIEKIGEGTYGVVYKGRHKTTGQVVAVKKIRLESEEEGVPSTAIREISLLKELRHPNIVSLQDVLMQDSRLYLIAEFLSMDLKKYLDSIPPGQYMDSSLVKSYLYQILQGIVFCHSRRVLHRDLKPQNLLIDDKGTIKLADFGLARAFGIPIRVYTHEVVTLWYRSPEVLLGSARYSTPVDIWSIGTIFAELATKKPLFHGDSEIDQLFRIFRALGTPNNEVWPEVESLQDYKNTFPKWKPGSLASHVKNLDENGLDLLSKMLIYDPAKRISGKMALNHPYFNDLDNQIKKM

SEQ ID NO: 7 - protein, 297 aa, Artificial Sequence
  Wild-type human CDK1 is mutated at position 75 (R75E) and position 80 (F80G)
MEDYTKIEKIGEGTYGVVYKGRHKTTGQVVAMKKIRLESEEEGVPSTAIREISLLKELRHPNIVSLQDVLMQDSELYLIGEFLSMDLKKYLDSIPPGQYMDSSLVKSYLYQILQGIVFCHSRRVLHRDLKPQNLLIDDKGTIKLADFGLARAFGIPIRVYTHEVVTLWYRSPEVLLGSARYSTPVDIWSIGTIFAELATKKPLFHGDSEIDQLFRIFRALGTPNNEVWPEVESLQDYKNTFPKWKPGSLASHVKNLDENGLDLLSKMLIYDPAKRISGKMALNHPYFNDLDNQIKKM

SEQ ID NO: 8 - protein, 297 aa, Artificial Sequence
  Wild-type human CDK1 is mutated at position 75 (R75E) and position 80 (F80A)
MEDYTKIEKIGEGTYGVVYKGRHKTTGQVVAMKKIRLESEEEGVPSTAIREISLLKELRHPNIVSLQDVLMQDSELYLIAEFLSMDLKKYLDSIPPGQYMDSSLVKSYLYQILQGIVFCHSRRVLHRDLKPQNLLIDDKGTIKLADFGLARAFGIPIRVYTHEVVTLWYRSPEVLLGSARYSTPVDIWSIGTIFAELATKKPLFHGDSEIDQLFRIFRALGTPNNEVWPEVESLQDYKNTFPKWKPGSLASHVKNLDENGLDLLSKMLIYDPAKRISGKMALNHPYFNDLDNQIKKM

SEQ ID NO: 9 - protein, 292 aa, Artificial Sequence
  Wild-type murine CDK5 is mutated at position 80 (F80G)
MQKYEKLEKIGEGTYGTVFKAKNRETHEIVALKRVRLDDDDEGVPSSALREICLLKELKHKNIVRLHDVLHSDKKLTLVGEFCDQDLKKYFDSCNGDLDPEIVKSFLFQLLKGLGFCHSRNVLHRDLKPQNLLINRNGELKLADFGLARAFGIPVRCYSAEVVTLWYRPPDVLFGAKLYSTSIDMWSAGCIFAELANAGRPLFPGNDVDDQLKRIFRLLGTPTEEQWPAMTKLPDYKPYPMYPATTSLVNVVPKLNATGRDLLQNLLKCNPVQRISAEEALQHPYFSDFCPP

SEQ ID NO: 10 - protein, 292 aa, Artificial Sequence
  Wild-type murine CDK5 is mutated at position 80 (F80A)
MQKYEKLEKIGEGTYGTVFKAKNRETHEIVALKRVRLDDDDEGVPSSALREICLLKELKHKNIVRLHDVLHSDKKLTLVAEFCDQDLKKYFDSCNGDLDPEIVKSFLFQLLKGLGFCHSRNVLHRDLKPQNLLINRNGELKLADFGLARAFGIPVRCYSAEVVTLWYRPPDVLFGAKLYSTSIDMWSAGCIFAELANAGRPLFPGNDVDDQLKRIFRLLGTPTEEQWPAMTKLPDYKPYPMYPATTSLVNVVPKLNATGRDLLQNLLKCNPVQRISAEEALQHPYFSDFCPP

SEQ ID NO: 11 - protein, 292 aa, Artificial Sequence
  Wild-type human CDK5 is mutated at position 80 (F80G)
MQKYEKLEKIGEGTYGTVFKAKNRETHEIVALKRVRLDDDDEGVPSSALREICLLKELKHKNIVRLHDVLHSDKKLTLVGEFCDQDLKKYFDSCNGDLDPEIVKSFLFQLLKGLGFCHSRNVLHRDLKPQNLLINRNGELKLADFGLARAFGIPVRCYSAEVVTLWYRPPDVLFGAKLYSTSIDMWSAGCIFAELANAGRPLFPGNDVDDQLKRIFRLLGTPTEEQWPSMTKLPDYKPYPMYPATTSLVNVVPKLNATGRDLLQNLLKCNPVQRISAEEALQHPYFSDFCPP

SEQ ID NO: 12 - protein, 292 aa, Artificial Sequence
  Wild-type human CDK5 is mutated at position 80 (F80A)
MQKYEKLEKIGEGTYGTVFKAKNRETHEIVALKRVRLDDDDEGVPSSALREICLLKELKHKNIVRLHDVLHSDKKLTLVAEFCDQDLKKYFDSCNGDLDPEIVKSFLFQLLKGLGFCHSRNVLHRDLKPQNLLINRNGELKLADFGLARAFGIPVRCYSAEVVTLWYRPPDVLFGAKLYSTSIDMWSAGCIFAELANAGRPLFPGNDVDDQLKRIFRLLGTPTEEQWPSMTKLPDYKPYPMYPATTSLVNVVPKLNATGRDLLQNLLKCNPVQRISAEEALQHPYFSDFCPP

(19) United States
(12) Patent Application Publication
(10) Pub. No.: US 2019/0185906 A1
(43) Pub. Date: Jun. 20, 2019

(54) IN SITU VISUALIZATION OF KINASE ACTIVITY

(71) Applicant: DANA-FARBER CANCER INSTITUTE, INC., BOSTON, MA (US)

(72) Inventors: Peter Sicinski, Waban, MA (US); Wojciech Michowski, Boston, MA (US)

(21) Appl. No.: 16/326,282

(22) PCT Filed: Aug. 17, 2017
(86) PCT No.: PCT/US17/47297

§ 371 Date: Feb. 18, 2019

Related U.S. Application Data
(60) Provisional application No. 62/377,262, filed on Aug. 19, 2016.

(51) Int. Cl. C12Q 1/48

(52) U.S. Cl. CPC C12Q 1/485

(57) ABSTRACT

Kinases can be engineered to utilize an ATP analog that is not readily utilized by wild-type kinases by introducing a mutation in the ATP-binding pocket. However, application of this method has been limited by the membrane impermeability of the ATP analog. Provided herein are methods for in situ visualization of substrates of an analog-sensitive kinase, the method comprising a mild fixation step. Also provided herein are kits comprising a fixative, an ATP analog, and an agent for detecting the substrates modified by the ATP analog.

### RELATED APPLICATIONS

This application claims the benefit of U.S. Provisional Application Nos. 62/377,262, filed Aug. 19, 2016, which is incorporated by reference herein in its entirety.

### GOVERNMENT SUPPORT

This invention was made with government support under grants RO1 CA083688 and RO1 CA132740 awarded by the National Institutes of Health (NIH). The government has certain rights in the invention.

### BACKGROUND

Phosphorylation is one of the most important protein modifications in signal transduction. Protein phosphorylation is regulated by protein kinases, which are under complex and dynamic regulations by other cellular apparatus. Methods have been developed to examine or monitor protein kinase activities, such as detecting phosphorylation of a known substrate protein. However, not all kinases have known substrates under a given condition, and detection of a phosphorylated protein is often limited by the availability of a potent and specific antibody. A more universal method is in need.

Shokat et al. pioneered a universal approach to resolve the problem. They introduced a mutation of a bulky gatekeeper residue in the ATP-binding pocket of a kinase, allowing this kinase to utilize a bulky ATP analog that is not readily utilized by wild-type kinases. This approach was described in WO1998035048, which is incorporated by reference herein in its entirety. Because nearly all kinases have bulky gatekeeper residues, this method is applicable to almost the entire kinome, although about 30% of the kinome does not tolerate gatekeeper mutations and requires a second-site suppressor mutation (Zhang et al., Nature Methods 2:435-441, incorporated by reference herein in its entirety). This method allows identification and quantification of proteins phosphorylated by a specific kinase in a homogenate, lysate or extract from cells or tissues.

However, the Shokat method requires a membrane permeabilization step due to the impermeability of bulky ATP analogs. Upon membrane permeabilization, cellular and intracellular architectures are disrupted and the native biological context fails to be maintained. Therefore, single-cell detection of kinase activity, which is commonly desired in state-of-the-art biological and biomedical research, has not been enabled in the original Shokat method. Moreover, this method could identify artificial kinase-substrate relationships which are present only in the in vitro biochemical system. Accordingly, there is a need for methods useful for in situ detection of kinase activity while maintaining the biological context.

### SUMMARY OF THE INVENTION

The instant disclosure provides methods for in situ detection of protein substrates of an analog-sensitive kinase. The methods are particularly useful for identification of kinase activity in cells in culture and within tissues at subcellular level at various physiological and pathological conditions, or for quantification of overall kinase activity at cellular or subcellular levels. Kits comprising agents for using the methods are also provided.

An aspect of the invention provides a method for in situ visualization of kinase activity in a sample comprising a kinase, the method comprising: (a) incubating the sample with a fixative; (b) incubating the sample with an ATP analog, such that the kinase accepts the ATP analog as a phosphate donor substrate, such that the γ-phosphate of the ATP analog comprises a transferrable label; and (c) detecting the transferrable label.

In various embodiments of the method, the fixative comprises an aldehyde. For example, the aldehyde is formaldehyde. In various embodiments of the method, the concentration of formaldehyde is between about 1% and about 10%. In related embodiments, the concentration of formaldehyde is between about 3% and about 5%. In a related embodiment, the concentration of formaldehyde is about 4%.

In various embodiments of the method, the sample is incubated with the fixative for about 10 minutes or shorter. In various embodiments, the sample is incubated with the fixative for about 5 minutes.

In various embodiments of the method, the fixative comprises an alcohol. For example, the alcohol is methanol or ethanol.

In various embodiments of the method, the ATP analog is a derivative of ATP having a substitution group comprising at least three carbon atoms covalently attached to the adenine group of the ATP. In various embodiments, the substitution group is attached to the N6 position of the ATP.

In various embodiments of the method, the ATP analog is selected from the group consisting of N6-furfuryladenosine-5′-O-(3-thiotriphosphate), N6-(cyclopentyl)ATP, N6-(cyclopentyloxy)ATP, N6-(cyclohexyl)ATP, N6-(cyclohexyloxy)ATP, N6-(benzyloxy)ATP, N6-(pyrolidino)ATP, N6-(ippperidino)ATP, N6-(2-phenylethyl)adenosine-5′-O-(3-thiotriphosphate) and N6-phenyladenosine-5′-O-(3-thiotriphosphate).

In various embodiments of the method, the substitution group is N6-furfuryladenosine-5′-O-(3-thiotriphosphate).

In various embodiments of the method, the transferrable label is a thiophosphate. In a related embodiment, detecting the transferrable label comprises alkylating the thiophosphate under suitable conditions to form a thiophosphoester. In various embodiments, the thiophosphoester comprises a detectable moiety. For example, the detectable moiety is selected from the group consisting of a fluorophore, an electron dense moiety, and a moiety specifically binding to a binding protein. In various embodiments, the detectable moiety is a moiety specifically binding to a binding protein, wherein detecting the transferrable label further comprises incubating the sample with the binding protein.

In various embodiments of the method, the binding protein is an anti-thiophosphoester antibody.

In various embodiments of the method, the detectable moiety is a biotin, and wherein the binding protein is avidin or a homolog thereof.

In various embodiments of the method, the suitable conditions comprise an acidic condition. In various embodiments, the acidic condition has a pH of 6.0 or lower. For example, the acidic condition has a pH of 5.0 or lower. For example, the acidic condition has a pH of about 4.0.

In various embodiments of the method, the transferrable label comprises a first click chemistry handle. For example, the first click chemistry handle is an azido group or a propargyl group.

In various embodiments of the method, detecting the transferrable label comprises contacting the first click chemistry handle with a second click chemistry handle. For example, the second click chemistry handle comprises a detectable moiety. In various embodiments, the detectable moiety is selected from the group consisting of a fluorophore, an electron dense moiety, and a moiety specifically binding to a binding protein.

In various embodiments of the method, the transferrable label is an uncommon isotope. For example, the uncommon isotope is an isotope of phosphorus, oxygen or hydrogen.

In various embodiments, the method further comprises a step of quenching a thiol group prior to step (b) described above, i.e., incubating the sample with an ATP analog, such that the kinase accepts the ATP analog as a phosphate donor substrate.

In various embodiments, the method further comprises a step of stopping kinase reaction after step (b) described above. In various embodiments, the kinase is selected from the group consisting of CDC7, AURKA, SRC, TTK, CDK9, CDK12, PLK4, MST3, ALK7, ROCK2, PKD, RET, EGFR, CDK7, ATM, EPHB1, EPHB2, EPHB3, PRKCI, NDR1, AMPKA2, ERK2, JAK1, JAK3, ZAP70, PRKCE, AKT, AURKB, CAMK2, CDK2, CSK, GRK2, JNK2, LCK, MEK1, PKA, PKCD, PLK1, RAFT, SAD1, SYK, TRKA, TRKB, TRKC, and JNK1. In various embodiments, the kinase comprises at least one amino acid substitution in the kinase domain.

In various embodiments of the method, the kinase comprises an amino acid sequence at least 80% identical to a sequence selected from the group consisting of SEQ ID NOs: 1-12.

In various embodiments of the method, the sample comprises a cell. In various embodiments, the sample comprises a tissue or an organ.

An aspect of the invention provides a kit comprising: (a.) a fixative; (b.) an ATP analog, wherein the γ-phosphate of the ATP analog comprises a transferrable label; and (c.) one or more agents for detecting the transferrable label.

In various embodiments of the kit, the fixative comprises an aldehyde. For example, the aldehyde is formaldehyde.

In various embodiments of the kit, the concentration of formaldehyde is between about 1% and about 10%. In a related embodiment, the concentration of formaldehyde is between about 3% and about 5%. In a related embodiment, the concentration of formaldehyde is about 4%.

In various embodiments, the kit further comprises an instruction to incubate a sample with the fixative for about 10 minutes or shorter. In various embodiments, the instruction instructs to incubate a sample with the fixative for about 5 minutes.

In various embodiments of the kit, the fixative comprises an alcohol.

In various embodiments of the kit, the ATP analog is a derivative of ATP having a substitution group comprising at least three carbon atoms covalently attached to the adenine group of the ATP. For example, wherein the substitution group is attached to the N6 position of the ATP.

In various embodiments of the kit, the substitution group is selected from the group consisting of N6-furfuryladenosine-5′-O-(3-thiotriphosphate), N6-(cyclopentyl)ATP, N6-(cyclopentyloxy)ATP, N6-(cyclohexyl)ATP, N6-(cyclohexyloxy)ATP, N6-(benzyloxy)ATP, N6-(pyrolidino)ATP, N6-(ippperidino)ATP, N6-(2-phenylethyl)adenosine-5′-O-(3-thiotriphosphate) and N6-phenyladenosine-5′-O-(3-thiotriphosphate). For example, the substitution group is N6-furfuryladenosine-5′-O-(3-thiotriphosphate).

In various embodiments of the kit, the transferrable label is a thiophosphate. In various embodiments, the one or more agents for detecting the transferrable label comprise an agent capable of alkylating the thiophosphate form a thiophosphoester. In various embodiments, the thiophosphoester comprises a detectable moiety. In various embodiments, the detectable moiety is selected from the group consisting of a fluorophore, an electron dense moiety, and a moiety specifically binding to a binding protein. In various embodiments, the detectable moiety is a moiety specifically binding to a binding protein, wherein the one or more agents for detecting the transferrable label further comprise the binding protein. For example, the binding protein is an anti-thiophosphoester antibody.

In various embodiments of the kit, the detectable moiety is a biotin, and wherein the binding protein is avidin or a homolog thereof.

In various embodiments of the kit, the one or more agents for detecting the transferrable label further comprise an acidic buffer for the alkylation reaction. In various embodiments of the kit, the pH of the acidic buffer is 6.0 or lower. In various embodiments, the pH of the acidic buffer is 5.0 or lower. In various embodiments, the pH of the acidic buffer is about 4.0.

In various embodiments of the kit, the transferrable label comprises a first click chemistry handle. For example, the first click chemistry handle is an azido group or a propargyl group.

In various embodiments of the kit, the one or more agents for detecting the transferrable label comprise a second click chemistry handle. In a related embodiment, the second click chemistry handle comprises a detectable moiety. In various embodiments, the detectable moiety is selected from the group consisting of a fluorophore, an electron dense moiety, and a moiety specifically binding to a binding protein.

In various embodiments, the transferrable label is an uncommon isotope. For example, the transferrable label is an uncommon isotope of phosphorus, oxygen or hydrogen.

In various embodiments, the kit further comprises an agent for quenching a thiol group.

In various embodiments, the kit further comprises an agent for stopping kinase reaction.

### BRIEF DESCRIPTION OF THE DRAWINGS

FIG. 1 is a diagram showing a method for detecting substrates of an analog-sensitive (AS) kinase. A kinase of interest is engineered to comprise an analog-sensitive mutation, so that the kinase is capable of using an ATP with a substitution group covalently attached to the adenine group of the ATP (bulky ATP) as a substrate. All other kinases are not able to use this bulky ATP. The bulky ATP is additionally modified to contain sulfur in the gamma phosphate, thereby being denoted as “bulky-γ-Thio-ATP.” The γ-thio phosphate is transferrable to a kinase substrate upon the AS kinase-catalyzed phosphorylation. The substrate is alkylated by p-Nitrobenzyl mesylate (PNBM) to create a semisynthetic epitope for an anti-thiophosphate ester antibody for detecting the substrates.

FIG. 2 is a photograph showing in situ detection of CDK1 substrates in cells during the metaphase of cell cycle. CDK1 was engineered to comprise an AS mutation. This mutant CDK1 was introduced by homologous recombination to V6.5 mouse embryonic stem cells. The cells were fixed by 4% formaldehyde for 5 minutes, incubated with 100 μM N6-furfuryladenosine-5′-O-(3-thiotriphosphate) for 20 minutes in the presence of 0.1% Triton X-100 and alkylated by 1 mM PNBM for 15 minutes. DNA was stained with Hoechst and the CDK1 substrates were stained with an anti-thiophosphoester antibody.

FIG. 3 is a series of photographs showing in situ detection of CDK1 substrates in embryonic stem cells. CDK1 was engineered to comprise an AS mutation (M32V, F80G) and this mutant CDK1 was substituted for the wild-type CDK1 in both alleles in embryonic stem cells. Wild-type (parts B and D) and the CDK1 AS mutant (parts A and C) cells were fixed by 4% formaldehyde for 5 minutes, incubated by 100 uM N6-furfuryladenosine-5′-O-(3-thiotriphosphate) and 0.1% Triton X-100 for 20 minutes, and alkylated by 1 mM PNBM for 15 minutes. DNA was stained with Hoechst and the CDK1 substrates were stained with an anti-thiophosphoester antibody. Parts A and B show a lower magnification and Parts C and D show a higher magnification of the images.

FIG. 4 is a series of photographs showing in situ detection of CDK5 substrates in neurons. CDK5 was engineered to comprise an AS mutation (F80G) and this mutant CDK5 was substituted for the wild-type CDK5 in both alleles in mice. Freshly harvested brains from adult wild-type (parts B and D) or the CDK5 AS mutant (parts A and C) mice were frozen and cut into 20 μm sections. The sections were fixed by 4% formaldehyde for 5 minutes, incubated by 100 uM N6-furfuryladenosine-5′-O-(3-thiotriphosphate) and 0.1% Triton X-100 for 20 minutes, and alkylated by 1 mM PNBM for 15 minutes. DNA was stained with Hoechst and the CDK5 substrates were stained with an anti-thiophosphoester antibody. Parts A and B show a lower magnification and Parts C and D show a higher magnification of the images.

FIG. 5 is a flowchart showing optimized steps and conditions for in situ visualization of substrates of a kinase of interest.

### DETAILED DESCRIPTION

It is to be understood that the methods described in this disclosure are not limited to particular methods and experimental conditions disclosed herein; as such methods and conditions may vary. It is also to be understood that the terminology used herein is for the purpose of describing particular embodiments only, and is not intended to be limiting.

Furthermore, the experiments described herein, unless otherwise indicated, use conventional molecular and cellular biological and immunological techniques within the skill of the art. Such techniques are well known to the skilled worker, and are explained fully in the literature. See, e.g., Ausubel, et al., ed., Current Protocols in Molecular Biology, John Wiley & Sons, Inc., NY, N.Y. (1987-2008), including all supplements, Molecular Cloning: A Laboratory Manual (Fourth Edition) by M R Green and J. Sambrook and Harlow et al., Antibodies: A Laboratory Manual, Chapter 14, Cold Spring Harbor Laboratory, Cold Spring Harbor (2013, 2^{nd} edition).

### I. Definitions

Unless otherwise defined herein, scientific and technical terms used herein have the meanings that are commonly understood by those of ordinary skill in the art. In the event of any latent ambiguity, definitions provided herein take precedent over any dictionary or extrinsic definition. Unless otherwise required by context, singular terms shall include pluralities and plural terms shall include the singular. The use of “or” means “and/or” unless stated otherwise. The use of the term “including”, as well as other forms, such as “includes” and “included”, is not limiting.

Generally, nomenclatures used in connection with cell and tissue culture, molecular biology, immunology, microbiology, genetics and protein and nucleic acid chemistry and hybridization described herein are those well-known and commonly used in the art. The methods and techniques provided herein are generally performed according to conventional methods well known in the art and as described in various general and more specific references that are cited and discussed throughout the present specification unless otherwise indicated. Enzymatic reactions and purification techniques are performed according to manufacturer's specifications, as commonly accomplished in the art or as described herein. The nomenclatures used in connection with, and the laboratory procedures and techniques of, analytical chemistry, synthetic organic chemistry, and medicinal and pharmaceutical chemistry described herein are those well-known and commonly used in the art. Standard techniques are used for chemical syntheses, chemical analyses, pharmaceutical preparation, formulation, and delivery, and treatment of patients.

That the disclosure may be more readily understood, select terms are defined below.

As used herein, the term “in situ” refers to detecting a signal from a molecule wherein the molecule is in its native location in an organelle, a cell, a tissue, an organ or an organism. In certain embodiment, the molecule is in its native location in an organelle while the cellular structure is not maintained. In certain embodiments, the molecule is in its native location in a cell wherein the structure of a tissue or an organ comprising the cell is not maintained. In certain embodiments, the molecule is in its native location in a tissue or in an organ wherein the structure of the organism comprising the tissue or the organ is not maintained.

As used herein, the term “kinase” refers to an enzyme capable of catalyzing the transfer of a phosphate group from a donating molecule to a substrate. In certain embodiments, the donating group is an adenosine triphosphate (ATP) or an ATP analog. In certain embodiments, the substrate is a protein.

As used herein, the term “fixative” refers to an agent capable of preserving the location of a molecule in an organelle, a cell, a tissue, an organ or an organism for in situ analysis, or an agent, a process or a device capable of generating thereof. Non-limiting examples include an aldehyde and an alcohol.

As used herein, the term “aldehyde” refers to a chemical compound comprising an aldehyde group depicted as below:

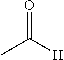

As used herein, the term “alcohol” refers to a chemical compound comprising an alcohol group depicted as below:

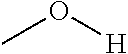

As used herein, the term “ATP analog” refers to a compound derived from adenosine-5′-triphosphate (ATP) or guanosine triphosphate (GTP). In certain embodiments, the ATP analog is a derivative of ATP having a substitution group comprising at least three carbon atoms covalently attached to the adenine group of the ATP. In certain embodiments, the ATP analog is a derivative of GTP having a substitution group comprising at least three carbon atoms covalently attached to the guanine group of the ATP.

As used herein, the term “N6 position of the ATP” refers to the position labeled as 6 in an adenine group as depicted below:

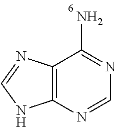

As used herein, the term “transferrable label” refers to a moiety encompassed in or attached to an atom of the γ-phosphate or a derivative thereof of the ATP analog. Non-limiting examples include a sulfur or an azide group attached to γ-phosphorus, and an isotope of phosphorus, oxygen or hydrogen in the γ-phosphate.

As used herein, the term “%” refers to mass percent (weight percent) or mass to volume percent. For instance, the term “4%” refers to 4 grams of solute per 100 grams or 100 ml of solution. In some embodiments, the solvent is water. In some embodiments, the solvent comprises water and a buffer. In some embodiments, the solvent comprises phosphate-buffered saline.

As used herein, the term “detectable moiety” refers to a moiety which can be detected with an imaging method, or a moiety which can be converted, modified, or conjugated to a moiety which can be detected with an imaging method. Non-limiting examples include a fluorophore, an electron dense moiety, and a moiety specifically binding to a binding protein.

As used herein, the term “acidic condition” refers to an aqueous condition at a temperature wherein the pH is lower than the neutral pH at the temperature. At 25° C., the neutral pH is 7.0. At 37° C., the neutral pH is 6.8.

As used herein, the term “click chemistry” refers to a chemical philosophy introduced by K. Barry Sharpless of The Scripps Research Institute, describing chemistry tailored to generate covalent bonds quickly and reliably by joining small units comprising reactive groups together (see, Kolb, Finn and Sharpless (2001) Angewandte Chemie International Edition 40: 2004-2021; Evans (2007) Australian Journal of Chemistry 60: 384-395, and Joerg Lahann (2009) Click Chemistry for Biotechnology and Materials Science, John Wiley & Sons Ltd, ISBN 978-0-470-69970-6, the contents of each of which are incorporated herein by reference in its entirety). Click chemistry does not refer to a specific reaction, but to a concept including, but not limited to, reactions that mimic reactions found in nature. In certain embodiments, click chemistry reactions are modular, wide in scope, give high chemical yields, generate inoffensive byproducts, are stereospecific, exhibit a large thermodynamic driving force to favor a reaction with a single reaction product, and/or can be carried out under physiological conditions. In certain embodiments, a click chemistry reaction exhibits high atom economy, can be carried out under simple reaction conditions, use readily available starting materials and reagents, uses no toxic solvents or use a solvent that is benign or easily removed (preferably water), and/or provides simple product isolation by non-chromatographic methods (crystallization or distillation). In certain embodiments, the click chemistry reaction is a [3+2] cycloaddition (e.g., an azide-alkyne cycloaddition, an azide-alkene cycloaddition). In certain embodiments, the click chemistry reaction is a [4+2] cycloaddition (e.g., a Diels-Alder cycloaddition between an alkene and a diene, a tetrazine or tetrazole).

As used herein, the term “click chemistry handle” refers to a conjugate reactant, or a reactive group, that can partake in a click chemistry reaction. In certain embodiments, the click chemistry handle is an alkyne (e.g., a terminal alkyne). In certain embodiments, the click chemistry handle is an azide.

As used herein, the term “uncommon isotope” refers to a chemical element which has the same number of protons and a different number of neutrons from the most abundant isotope of the element in nature. In certain embodiments, the uncommon isotope is ³²P, ²H, ³H, ¹⁸O, ¹⁸F or ³⁵S.

As used herein, the term “quenching a thiol group” refers to incubating a sample comprising a thiol group with an agent that reacts with the thiol group, wherein the product of the reaction does not contain a thiol group. Non-limiting examples of the agent that reacts with a thiol group include an electrophile, e.g., iodoacetamide and N-ethylmaleimide.

### II. Methods for In Situ Visualization of Kinase Activity

In certain aspects, the instant disclosure provides a method for in situ visualization of kinase activity in a sample comprising a kinase, the method comprising: incubating the sample with a fixative; incubating the sample with an ATP analog, wherein the kinase accepts the ATP analog as a phosphate donor substrate, wherein the γ-phosphate of the ATP analog comprises a transferrable label; and detecting the transferrable label.

In certain embodiments, the fixative comprises an aldehyde. In certain embodiments, the aldehyde is selected from the group consisting of formaldehyde, acetaldehyde and glutaraldehyde. In certain embodiments, the aldehyde is formaldehyde. In certain embodiments, the concentration of formaldehyde in the fixative is no greater than 10% (e.g., between about 1% and about 10%). In certain embodiments, the concentration of formaldehyde in the fixative is between about 3% and about 5%. In certain embodiments, the concentration of formaldehyde in the fixative is about 4%. In certain embodiments, the formaldehyde is dissolved in an aqueous solution. In certain embodiments, the formaldehyde is prepared by dissolving paraformaldehyde (PFA) in a solvent. In certain embodiments, the solvent is water. In some embodiments, the solvent comprises water and a buffer. In some embodiments, the solvent comprises phosphate-buffered saline.

In certain embodiments, the fixative comprises an organic solvent. In certain embodiments, the organic solvent is an alcohol. In certain embodiments, the alcohol is selected from the group consisting of methanol and ethanol. In certain embodiments, the organic solvent is acetone. In certain embodiments, the fixative is a formalin-free fixative. In certain embodiments, the formalin-free fixative is Hepes-glutamic acid buffer mediated Organic solvent Protection Effect (HOPE) technique.

In certain embodiments, the sample is incubated with the fixative for about 10 minutes or shorter. In certain embodiments, the sample is incubated with the fixative for at least 1 minute and at most 10 minutes. In certain embodiments, the sample is incubated with the fixative for about 5 minutes. In certain embodiments, the incubation occurs at room temperature. In certain embodiments, the incubation occurs at about 37° C., about 30° C., about 25° C., about 20° C., about 15° C., about 10° C., or about 4° C.

In certain embodiments, the ATP analog is a derivative of ATP or GTP. In certain embodiments, the ATP analog has a substitution group covalently attached to the adenine group of the ATP or the guanine group of the GTP. In certain embodiments, the substitution group comprises at least three carbon atoms. In certain embodiments, the substitution group comprises at least one cyclic. In certain embodiments, the substitution group comprises at least one aryl group. In certain embodiments, the substitution group is attached to the N6 position of the ATP. In certain embodiments, the ATP analog is selected from the group consisting of N6-furfuryladenosine-5′-O-(3-thiotriphosphate) (6-Fu-ATP-γ-S), N6-(cyclopentyl)ATP, N6-(cyclopentyloxy)ATP, N6-(cyclohexyl)ATP, N6-(cyclohexyloxy)ATP, N6-(benzyloxy)ATP, N6-(pyrolidino)ATP, N6-(ippperidino)ATP, N6-(2-phenylethyl)adenosine-5′-O-(3-thiotriphosphate) (6-PhEt-ATP-γ-S) and N6-phenyladenosine-5′-O-(3-thiotriphosphate) (6-Phe-ATP-γ-S). In certain embodiments, the ATP analog is 6-Fu-ATP-γ-S.

In certain embodiments, the transferrable label is a thiophosphate. In certain embodiments, detecting the transferrable label comprises alkylating the thiophosphate with an alkylating agent under suitable conditions to form a thiophosphoester. In certain embodiments, the alkylating agent comprises a nucleophilic group. In certain embodiments, the alkylating agent is p-Nitrobenzyl mesylate (PNBM). In certain embodiments, the suitable conditions comprise an acidic condition. In certain embodiments, the acidic condition has a pH of 6.0 or lower. In certain embodiments, the acidic condition has a pH of 5.0 or lower. In certain embodiments, the acidic condition has a pH of 4.0 or lower. In certain embodiments, the acidic condition has a pH in the inclusive range between 4.0 and 6.0. In certain embodiments, the acidic condition has a pH in the inclusive range between 4.0 and 5.0. In certain embodiments, the acidic condition has a pH in the inclusive range between 5.0 and 6.0.

In certain embodiments, the thiophosphoester comprises a detectable moiety. In certain embodiments, the detectable moiety is selected from the group consisting of a fluorophore, an electron dense moiety, and a moiety specifically binding to a binding protein. In certain embodiments, the detectable moiety is conjugated to the alkylating agent. In other embodiments, the detectable moiety is generated from the alkylation reaction. In one embodiment, the detectable moiety is a thiophosphoester group. In certain embodiments, the detectable moiety is a moiety specifically binding to a binding protein (e.g., an antibody), wherein detecting the transferrable label further comprises incubating the sample with the binding protein. In one embodiment, the antibody is an anti-thiophosphoester antibody. In certain embodiments, the detectable moiety is a biotin, and the binding protein is avidin or a homolog thereof. In certain embodiments, the alkylating agent is selected from the group consisting of N-iodoacetyl-N-biotinylhexylenediamine and (+)-biotinyl-iodoacetamidyl-3,6-dioxaoctanediamine. In certain embodiments, the avidin or homolog thereof is selected from the group consisting of avidin (e.g., comprising natural glycosylation), streptavidin, NeutrAvidin. In certain embodiments, the detectable moiety is a fluorophore. In certain embodiments, the fluorophore is selected from the group consisting of AF488 C5Meleimid and OregonGreen 488 Iodoacetamide.

In certain embodiments, the transferrable label comprises a first click chemistry handle. In certain embodiments, detecting the transferrable label comprises contacting the first click chemistry handle with a second click chemistry handle. In certain embodiments, the first click chemistry handle is an azido group and the second click chemistry handle is an alkyne (e.g., a terminal alkyne). In certain embodiments, the first click chemistry handle comprises a propargyl group and the second click chemistry group is an azide. In certain embodiments, the transferrable label is selected from the group consisting of (2-azidoethyl)phosphate, (((6-azidohexyl)amino)oxy)phosphate, and (((propargyl)amino)oxy)phosphate. In certain embodiments, the ATP analog is selected from the group consisting of γ-(2-azidoethyl)-ATP, γ-(6-azidohexyl)imido-ATP, and γ-(propargyl)imido-ATP, wherein a substitution group is optionally attached to the N6 position of the ATP. In certain embodiments, detecting the transferrable label comprises contacting the first click chemistry handle with the second click chemistry handle in the presence of a catalyst. In certain embodiments, the catalyst comprises Cu(I). In certain embodiments, the second click chemistry handle comprises a detectable label. In certain embodiments, the detectable label is selected from the group consisting of a fluorophore, an electron dense moiety, and a moiety specifically binding to an antibody. In certain embodiments, the detectable moiety is conjugated to the second click chemistry handle. In other embodiments, the detectable moiety is generated from the click chemistry reaction.

In certain embodiments, detecting the transferrable label comprises fluorescent microscopy. In certain embodiments, detecting the transferrable label comprises immunohistochemistry. In certain embodiments, detecting the transferrable label comprises electron microscopy. In certain embodiments, detecting the transferrable label comprises chromatography. In certain embodiments, detecting the transferrable label comprises electrophoresis. In certain embodiments, detecting the transferrable label comprises flow cytometry. In certain embodiments, detecting the transferrable label comprises immunoblotting. In certain embodiments, detecting the transferrable label further comprises reversing crosslinking wherein the sample was previously incubated with an aldehyde fixative. In certain embodiments, detecting the transferrable label further comprises a step of stopping the thiophosphate alkylation or click chemistry reaction. In certain embodiments, the thiophosphate alkylation reaction is stopped by an agent comprising an active thiol group (e.g., beta-mercaptoethanol, dithiothreitol). In certain embodiments, the click chemistry reaction is stopped by addition of an agent that reacts with a click chemistry handle, or an agent that reacts with a catalyst in the click chemistry reaction.

In certain embodiments, the transferrable label is an uncommon isotope. In certain embodiments, the transferrable label is an uncommon isotope of phosphorus, hydrogen, oxygen, fluorine or sulfer. In certain embodiments, the uncommon isotope is ³²P, ²H, ³H, ¹⁸O, ¹⁸F or ³⁵S. In certain embodiments, detecting the transferrable label comprises nuclear imaging. In certain embodiments, the nuclear imaging comprises positron emission tomography. In certain embodiments, the nuclear imaging comprises magnetic resonance imaging.

In certain embodiments, the kinase is a wild-type kinase, wherein the ATP analog is different from ATP or GTP only at the γ position. In certain embodiments, the kinase comprises at least one amino acid substitution in the kinase domain. In certain embodiments, the kinase comprises a substitution of a small residue for a bulky gatekeeper residue in the kinase domain. In certain embodiments, the kinase further comprises a suppressor mutation that restores the activity compromised by the gatekeeper mutation. Non-limiting examples of the kinase include: AKT, ALK7, AMPK, ATM, AURKA, AURKB, BTK, CAMK2, CDK1, CDK12, CDK2, CDK5, CDK7, CDK9, CSK, EGFR, EPHB1, EPHB2, EPHB3, EPHB4, ERK2, GRK2, GSK3β, GSKα, JAK1, JAK3, JNK2, LCK, MAP3K5, MAPK14, MEK1, MET, MST3, NDR1, PDGFRB, PKA, PKCD, PKCE, PKD, PLK1, PLK4, PRKCI, RAF1, RET, ROCK2, SAD1, SRC, SYK, TRKA, TRKB, TRKC, TTK, and ZAP70. In certain embodiments, the kinase is or is derived from a human kinase, a murine kinase, or a kinase from a mammalian species. In certain embodiments, the kinase is CDK1 or CDK5. In certain embodiments, the kinase comprises an amino acid sequence selected from the group consisting of SEQ ID NOs: 1-12. In certain embodiments, kinase comprises an amino acid sequence at least 50%, at least 60%, at least 70%, at least 75%, at least 80%, at least 85%, at least 90%, at least 95%, or 100% identical to the amino acid sequence of a wild-type kinase, e.g., a wild-type human or murine CDC7, AURKA, SRC, TTK, CDK9, CDK12, PLK4, MST3, ALK7, ROCK2, PKD, RET, EGFR, CDK7, ATM, EPHB1, EPHB2, EPHB3, PRKCI, NDR1, AMPKA2, ERK2, JAK1, JAK3, ZAP70, PRKCE, AKT, AURKB, CAMK2, CDK2, CSK, GRK2, JNK2, LCK, MEK1, PKA, PKCD, PLK1, RAF1, SAD1, SYK, TRKA, TRKB, TRKC, JNK1.

Exemplary kinases are disclosed in Reference Nos. 1-37, the contents of which are incorporated by reference herein in their entirety.

In certain embodiments, the sample comprises a cell. In certain embodiments, the sample comprises a tissue or an organ. In certain embodiments, the sample is from an animal, e.g. a human, a mammal, a vertebrate or an invertebrate. In certain embodiments, the sample is from a plant, a fungus, a protist, an archaeon, a bacterium or a virus.

In certain embodiments, the sample is a monolayer of cells mounted on a support material, e.g., a coverslip. In certain embodiments, the sample is a tissue section. In certain embodiments, the sample is a population of cells in suspension. In one embodiment, detecting the transferrable label comprises flow cytometry analysis of the population of cells in suspension. In a particular embodiment, the population of cells is further labeled with one or more additional agents that recognize one or more molecules indicative of the cell type or status.

In certain embodiments, the method further comprises incubating the sample with a permeabilizing agent (e.g., an agent that permeabilizes the plasma membrane and optionally membrane enclosing or surrounding one or more intracellular organelles). In certain embodiments, the permeabilizing agent comprises Triton X-100, digitonin or saponin. In certain embodiments, the permeabilizing agent comprises 0.1% Triton X-100. In certain embodiments, the permeabilizing agent comprises 10-100 ug/ml digitonin. In certain embodiments, the sample is incubated with the peameabilizing agent before being incubated with the ATP analog. In certain embodiments, the sample is incubated with the permeabilizing agent and the ATP analog at the same time.

In certain embodiments, the method further comprises a step of quenching a thiol group prior to incubating the sample with the ATP analog. In certain embodiments, this step is conducted after the step of incubating the sample with a fixative. In certain embodiments, this step is conducted prior to or simultaneously with the step of incubating the sample with a fixative. In certain embodiments, the thiol group is an endogenous thiol group, e.g., the thiol group of a cysteine or cysteine residue. In certain embodiments, at least 10%, at least 20%, at least 30%, at least 40%, at least 50%, at least 60%, at least 70%, at least 80%, at least 90%, or 100% of the thiol groups in the sample is quenched.

In certain embodiments, the method further comprises a step of stopping kinase reaction after incubating the sample with the ATP analog. In certain embodiments, this step is conducted prior to or simultaneously with the step of detecting the transferrable label. In certain embodiments, stopping kinase reaction comprises changing a condition required for the kinase reaction. In certain embodiments, the condition is temperature, pH, or a molecule required for the kinase reaction (e.g., Mg²⁺). In certain embodiments, an agent (e.g., an acid, a base, or an agent that reacts with a required molecule) is added to the kinase reaction. In one embodiment, the agent that reacts with a required molecule is ethylenediaminetetraacetic acid (EDTA) or a salt thereof. In certain embodiments, the solution of the kinase reaction is removed from the sample, optionally followed by addition of an agent that changes a condition required for the kinase reaction. In certain embodiments, stopping kinase reaction comprises adding a second fixative. In certain embodiment, the second fixative is the same as the first fixative incubated with the sample prior to the kinase reaction, wherein the concentration of the second fixative is the same, higher or lower than the first fixative. In certain embodiments, the second fixative is different from the first fixative incubated with the sample prior to the kinase reaction. In certain embodiments, the second fixative causes protein crosslinking. In certain embodiments, the second fixative causes protein denaturation.

### III. Kits for In Situ Visualization of Kinase Activity

In certain aspects, the instant disclosure provides a kit comprising a fixative; an ATP analog, wherein the γ-phosphate of the ATP analog comprises a transferrable label; and one or more agents for detecting the transferrable label. In certain embodiments, the kit further comprises a permeabilizing agent (e.g., Triton X-100, digitonin, or saponin). In certain embodiments, the permeabilizing agent is 0.1% Triton X-100. In certain embodiments, the permeabilizing agent comprises 50 ug/ml digitonin. In certain embodiments, the permeabilizing agent and the ATP analog are provided in a single solution.

In certain embodiments, the fixative comprises an aldehyde. In certain embodiments, the aldehyde is selected from the group consisting of formaldehyde, acetaldehyde and glutaraldehyde. In certain embodiments, the aldehyde is formaldehyde. In certain embodiments, the concentration of formaldehyde in the fixative is no greater than 10% (e.g., between about 1% and about 10%). In certain embodiments, the concentration of formaldehyde in the fixative is between about 3% and about 5%. In certain embodiments, the concentration of formaldehyde in the fixative is about 4%. In certain embodiments, the formaldehyde is dissolved in an aqueous solution. In certain embodiments, the formaldehyde is prepared by dissolving paraformaldehyde (PFA) in a solvent. In certain embodiments, the solvent is water. In some embodiments, the solvent comprises water and a buffer. In some embodiments, the solvent comprises phosphate-buffered saline.

In certain embodiments, the fixative comprises an organic solvent. In certain embodiments, the organic solvent is an alcohol. In certain embodiments, the alcohol is selected from the group consisting of methanol and ethanol. In certain embodiments, the organic solvent is acetone. In certain embodiments, the fixative is a formalin-free fixative. In certain embodiments, the formalin-free fixative is Hepes-glutamic acid buffer mediated Organic solvent Protection Effect (HOPE) technique.

In certain embodiments, the ATP analog is a derivative of ATP or GTP. In certain embodiments, the ATP analog has a substitution group covalently attached to the adenine group of the ATP or the guanine group of the GTP. In certain embodiments, the substitution group comprises at least three carbon atoms. In certain embodiments, the substitution group comprises at least one cyclic. In certain embodiments, the substitution group comprises at least one aryl group. In certain embodiments, the substitution group is attached to the N6 position of the ATP. In certain embodiments, the ATP analog is selected from the group consisting of N6-furfuryladenosine-5′-O-(3-thiotriphosphate) (6-Fu-ATP-γ-S), N6-(cyclopentyl)ATP, N6-(cyclopentyloxy)ATP, N6-(cyclohexyl)ATP, N6-(cyclohexyloxy)ATP, N6-(benzyloxy)ATP, N6-(pyrolidino)ATP, N6-(ippperidino)ATP, N6-(2-phenylethyl)adenosine-5′-O-(3-thiotriphosphate) (6-PhEt-ATP-γ-S) and N6-phenyladenosine-5′-O-(3-thiotriphosphate) (6-Phe-ATP-γ-S). In certain embodiments, the ATP analog is 6-Fu-ATP-γ-S.

In certain embodiments, the transferrable label is a thiophosphate. In certain embodiments, the one or more agents for detecting the transferrable label comprise an agent capable of alkylating the thiophosphate to form a thiophosphoester. In certain embodiments, the agent capable of alkylating the thiophosphate comprises a nucleophilic group. In certain embodiments, the agent capable of alkylating the thiophosphate is p-Nitrobenzyl mesylate (PNBM). In certain embodiments, the one or more agents for detecting the transferrable label further comprise an acidic buffer for the alkylation reaction. In certain embodiments, the acidic buffer has a pH of 6.0 or lower. In certain embodiments, the acidic buffer has a pH of 5.0 or lower. In certain embodiments, the acidic buffer has a pH of 4.0 or lower. In certain embodiments, the acidic buffer has a pH in the inclusive range between 4.0 and 6.0. In certain embodiments, the acidic buffer has a pH in the inclusive range between 4.0 and 5.0. In certain embodiments, the acidic buffer has a pH in the inclusive range between 5.0 and 6.0. In certain embodiments, the one or more agents for detecting the transferrable label comprise iodoacetamide (IAM). In certain embodiments, the suitable conditions comprise an acidic buffer and IAM.

In certain embodiments, the thiophosphoester comprises a detectable moiety. In certain embodiments, the detectable moiety is selected from the group consisting of a fluorophore, an electron dense moiety, and a moiety specifically binding to a binding protein. In certain embodiments, the detectable moiety is conjugated to the alkylating agent. In other embodiments, the detectable moiety is generated from the alkylation reaction. In one embodiment, the detectable moiety is a thiophosphoester group. In certain embodiments, the detectable moiety is a moiety specifically binding to a binding protein (e.g., a first antibody), wherein the one or more agents for detecting the transferrable label comprise the binding protein (e.g., the first antibody). In certain embodiments, the first antibody is conjugated to a fluorescent moiety or an enzyme, e.g., horseradish peroxidase. In other embodiments, the one or more agents for detecting the transferrable label further comprise a secondary antibody that binds to the first antibody. In one embodiment, the first antibody is an anti-thiophosphoester antibody. In certain embodiments, the detectable moiety is a biotin, and the binding protein is avidin or a homolog thereof. In certain embodiments, the alkylating agent is selected from the group consisting of N-iodoacetyl-N-biotinylhexylenediamine and (+)-biotinyl-iodoacetamidyl-3,6-dioxaoctanediamine. In certain embodiments, the avidin or homolog thereof is selected from the group consisting of avidin (e.g., comprising natural glycosylation), streptavidin, NeutrAvidin. In certain embodiments, the detectable moiety is a fluorophore. In certain embodiments, the fluorophore is selected from the group consisting of AF488 C5Meleimid and OregonGreen 488 Iodoacetamide.

In certain embodiments, the transferrable label comprises a first click chemistry handle. is an azide group. In certain embodiments, the one or more agents for detecting the transferrable label comprise a second click chemistry handle. In certain embodiments, the first click chemistry handle is an azido group, and the second click chemistry handle is an alkyne, e.g., (a terminal alkyne). In certain embodiments, the first click chemistry handle comprises a propargyl group and the second click chemistry group is an azide. In certain embodiments, the transferrable label is selected from the group consisting of (2-azidoethyl)phosphate, (((6-azidohexyl)amino)oxy)phosphate, and (((propargyl)amino)oxy)phosphate. In certain embodiments, the ATP analog is selected from the group consisting of γ-(2-azidoethyl)-ATP, γ-(6-azidohexyl)imido-ATP, and γ-(propargyl)imido-ATP, wherein a substitution group is optionally attached to the N6 position of the ATP. In certain embodiments, the one or more agents for detecting the transferrable label further comprise a catalyst. In certain embodiments, the catalyst comprises Cu(I). In certain embodiments, the second click chemistry handle comprises a detectable label. In certain embodiments, the detectable label is selected from the group consisting of a fluorophore, an electron dense moiety, and a moiety specifically binding to an antibody. In certain embodiments, the detectable moiety is conjugated to the second click chemistry handle. In other embodiments, the detectable moiety is generated from the click chemistry reaction.

In certain embodiments, the one or more agent for detecting the transferrable label further comprises an agent for stopping the thiophosphate alkylation or click chemistry reaction. In certain embodiments, the agent for stopping the thiophosphate alkylation reaction comprises an active thiol group (e.g., beta-mercaptoethanol, dithiothreitol). In certain embodiments, the agent for stopping the click chemistry reaction is capable of reacting with a click chemistry handle or with a catalyst in the click chemistry reaction.

In certain embodiments, the transferrable label is an uncommon isotope. In certain embodiments, the transferrable label is an uncommon isotope of phosphorus, hydrogen, oxygen, fluorine or sulfer. In certain embodiments, the uncommon isotope is ³²P, ²H, ³H, ¹⁸O, ¹⁸F or ³⁵S. In certain embodiments, the kit does not comprise one or more agents for detecting the transferrable label wherein the transferrable label is an uncommon isotope.

In certain embodiments, the kit further comprises an agent for quenching a thiol group. In certain embodiments, the thiol group is an endogenous thiol group, e.g., the thiol group of a cysteine or cysteine residue. In certain embodiments, the agent for quenching a thiol group and the fixative are comprised in different solutions. In certain embodiments, the agent for quenching a thiol group and the fixative are comprised in the same solution.

In certain embodiments, the kit further comprises an agent for stopping kinase reaction. In certain embodiments, the agent for stopping kinase reaction and the agent for detecting the transferrable label are comprised in different solutions. In certain embodiments, the agent for stopping kinase reaction and the agent for detecting the transferrable label are comprised in the same solution. In certain embodiments, the agent for stopping kinase reaction is capable of changing a condition required for the kinase reaction. In certain embodiments, the condition is temperature, pH, or a molecule required for the kinase reaction (e.g., Mg²⁺). In certain embodiments, the agent for stopping kinase reaction comprises an acid, a base, or an agent that reacts with a required molecule. In one embodiment, the agent that reacts with a required molecule is ethylenediaminetetraacetic acid (EDTA) or a salt thereof. In certain embodiments, the agents for stopping kinase reaction comprises a second fixative. In certain embodiment, the second fixative is the same as the first fixative in the kit, wherein the concentration of the second fixative is the same, higher or lower than the first fixative. In certain embodiments, the second fixative is different from the first fixative. In certain embodiments, the second fixative causes protein crosslinking. In certain embodiments, the second fixative causes protein denaturation.

In certain embodiments, the kit further comprises instructions for use. In certain embodiments, the instructions are provided as an insert sheet. In certain embodiments, the instructions are provided as a computer-readable form carried on a device or transmitted or obtainable from a location on the Internet.

It will be readily apparent to those skilled in the art that other suitable modifications and adaptations of the methods described herein may be made using suitable equivalents without departing from the scope of the embodiments disclosed herein. Having now described certain embodiments in detail, the same will be more clearly understood by reference to the following examples, which are included for purposes of illustration only and are not intended to be limiting.

### EXAMPLES

The present invention is further illustrated by the following examples which should not be construed as further limiting.

### Example 1: Detection of Substrates of a Kinase of Interest by Generating an Analog-Sensitive (AS) Mutation in the Kinase

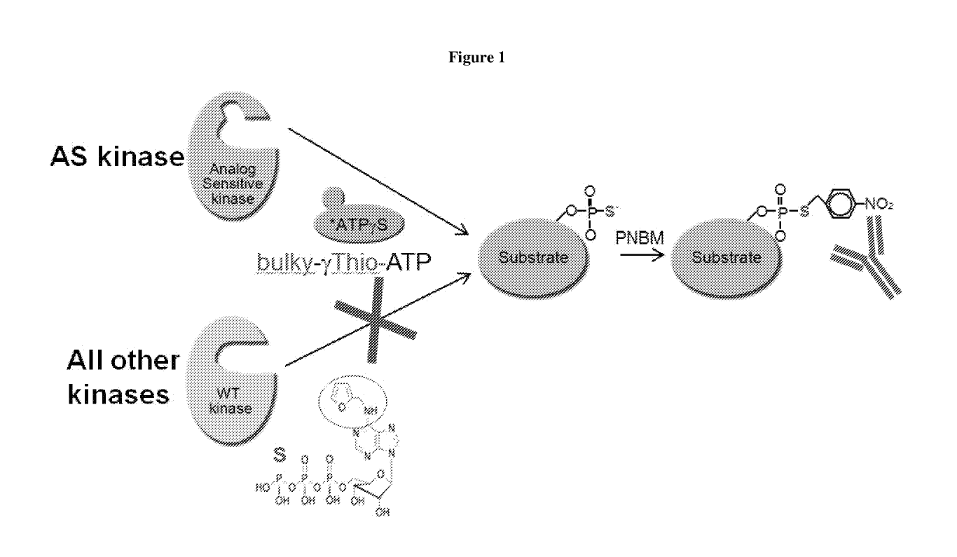

This example describes a method for detecting substrates of a kinase of interest. As shown in FIG. 1, the ATP-binding pocket of a kinase of interest was modified by a single amino acid substitution to generate an analog-sensitive (AS) enzyme which can accommodate a “bulky” analog of ATP. These analogs cannot be utilized by wild-type kinases. The bulky ATP was additionally modified to contain sulfur in the gamma phosphate, thereby being denoted “bulky-γ-Thio-ATP.” Cells or organisms e.g. mice were engineered to express the AS version of the kinase of interest. The AS kinase used bulky-γ-Thio-ATP and incorporated thiophosphate moieties into its protein substrates. These thiophosphate groups were next alkylated with p-Nitrobenzyl mesylate (PNBM) to create a semisynthetic epitope for an anti-thiophosphate ester antibody for detecting the substrates.

This method, modified from Shokat's invention as described in PCT patent application published as WO1998035048, is limited by the membrane impermeability of bulky-γ-Thio-ATP. Accordingly, this method has been used only with purified proteins or homogenate, lysate or extract from cells or tissues, wherein the natural kinase-substrate interaction may be disrupted by the destruction of biological context and the special organization of the cell or tissue is not maintained.

### Example 2: Fixation of Cells in an In Vitro Culture for In Situ Visualization of Kinase Substrates

This example describes a method of fixing cells in an in vitro culture and visualizing substrates of an AS kinase in the cells, which resolved the problem in Example 1.

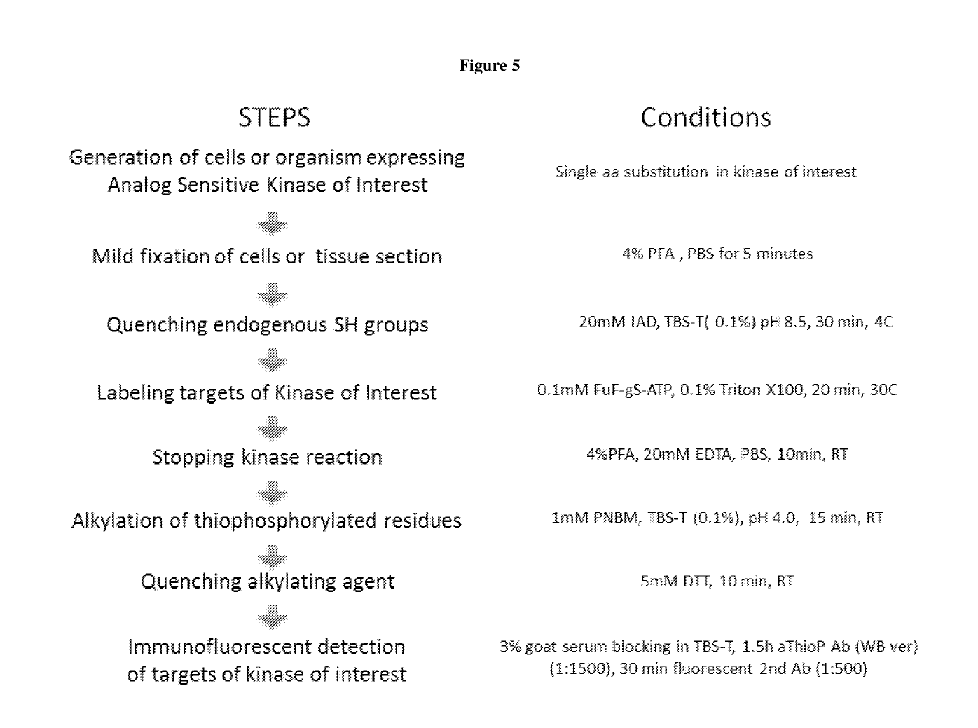

Murine CDK1 was engineered to comprise an AS mutation comprising M32V and F80G substitutions and this mutant CDK1 was substituted for the wild-type CDK1 in both alleles in murine embryonic stem cells. The sequence of the mutant CDK1 is shown in SEQ ID NO: 1, and other mutant CDK1 proteins comprising SEQ ID NOs: 2-4 are expected to function similarly Wild-type or AS mutant murine embryonic stem cells were cultured on glass coverslips and processed according to the steps in FIG. 5. The cells were fixed with 4% formaldehyde at room temperature for 5 minutes. The endogenous thiols were quenched with 20 mM iodoacetamide at 4° C. for 30 minutes. The cells were incubated with 100 μM furfuryladenosine-5′-O-(3-thiotriphosphate) in the presence of 0.1% Triton X-100 and a protease and phosphatase inhibitor cocktail at 30° C. for 20 minutes, thereby allowing thiophosphorylation of substrates of the AS kinase. The kinase reaction was stopped by incubating the cells with 20 mM EDTA and 4% formaldehyde in PBS for 10 minutes at room temperature. Thiophosphorylated residues of the substrates were subsequently alkylated with 1 mM PNBM at pH 4.0 for 15 minutes in the presence of 0.1% Triton X-100. The alkylation reaction was stopped by incubating the cells with 5 mM dithiothreitol (DTT) at room temperature for 10 minutes. The alkylated thiophosphate was visualized with an anti-thiophosphoester antibody and a secondary antibody coupled with AlexaFluor 594.

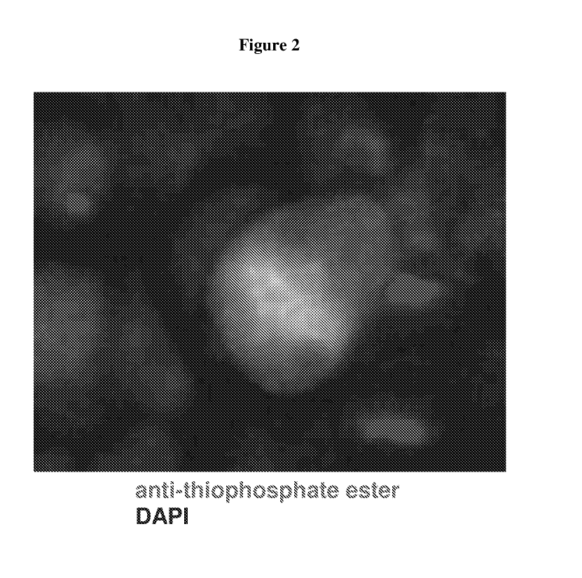

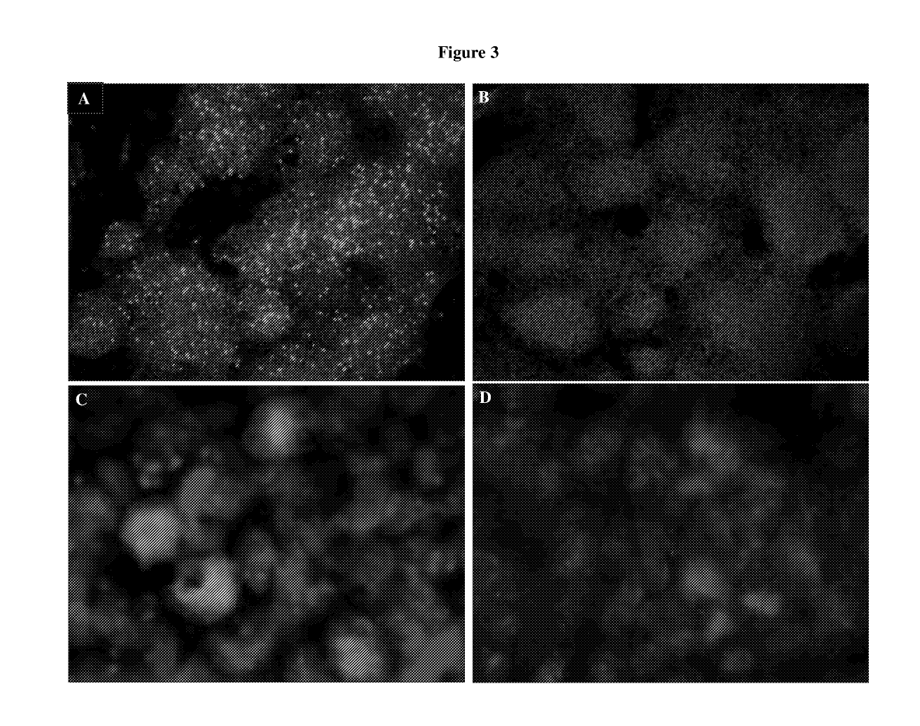

CDK1 activity was present in cells containing condensed chromosomes, which is consistent with known role of CDK1 in progression through mitosis (FIGS. 2 and 3). The specific signal was detected only in cells expressing the AS mutant kinase, verifying the kinase-substrate specificity under this condition.

Multiple fixation conditions were examined to determine the optimal duration of fixation. Without fixation, the permeabilization step with detergent caused destruction of cell morphology—even digitonin, a mild detergent, failed to maintain the cellular structure. Fixation with 4% formaldehyde for 1 minute was not sufficient for cells to adhere to the coverslip during subsequent steps. Fixation with 4% formaldehyde for 10 minutes resulted in visibly lower signal of AS kinase substrates as compared to a 5-minute fixation.

Two signal enhancement methods were tested. First, purified histone protein, a known substrate of CDK1, was added to the kinase reaction to provide a higher concentration of substrate in the proximity of CDK1 in fixed cells. Contrary to the expectation, the addition of histone decreased the signal intensity and altered the staining pattern. Second, only protease inhibitors but no phosphatase inhibitors were supplemented in the thiophosphorylation reaction, so that phosphatase activity might increase the amount of free serine and threonine residues. This second method failed to enhance the signal as expected.

Multiple alkylation conditions were examined to determine the optimal condition for reducing background staining, which may arise from non-specific alkylation of cysteine thiols. The level of background staining was assessed by performing the thiophosphorylation, alkylation and staining steps in wild-type cells which did not express an AS mutant kinase. Reducing the pH from 8.5 to 6, 5 or 4 resulted in significantly reduced background staining with no adverse effect on the signal strength. Addition of iodoacetamide after fixation in order to block endogenous cysteines thiols and before kinase reaction and alkylation further decreased the background staining.

### Example 3: Fixation of Brain Tissues for In Situ Visualization of Kinase Substrates

This example describes a method of fixing a tissue or an organ and visualizing substrates of an AS kinase in the tissue or organ, which resolved the problem in Example 1.

Murine CDK5 was engineered to comprise an AS mutation (F80G) and this mutant CDK5 was substituted for the wild-type CDK5 in both alleles in mice. The sequence of the mutant CDK5 is shown in SEQ ID NO: 9, and another mutant CDK1 protein comprising SEQ ID NO: 10 is expected to function similarly. Freshly harvested brains from adult wild-type or mutant mice were frozen and cut into 20 μm sections. The sections were mounted on glass coverslips and fixed with 4% formaldehyde for 5 minutes. The sections were then incubated with 100 μM furfuryladenosine-5′-O-(3-thiotriphosphate) in the presence of 0.1% Triton X-100 for 20 minutes, thereby allowing thiophosphorylation of substrates of the AS kinase. Thiophosphorylated substrates were alkylated with PNBM at pH 4.0 to enable detection with an anti-thiophosphoester antibody and a secondary antibody coupled with AlexaFluor 594.

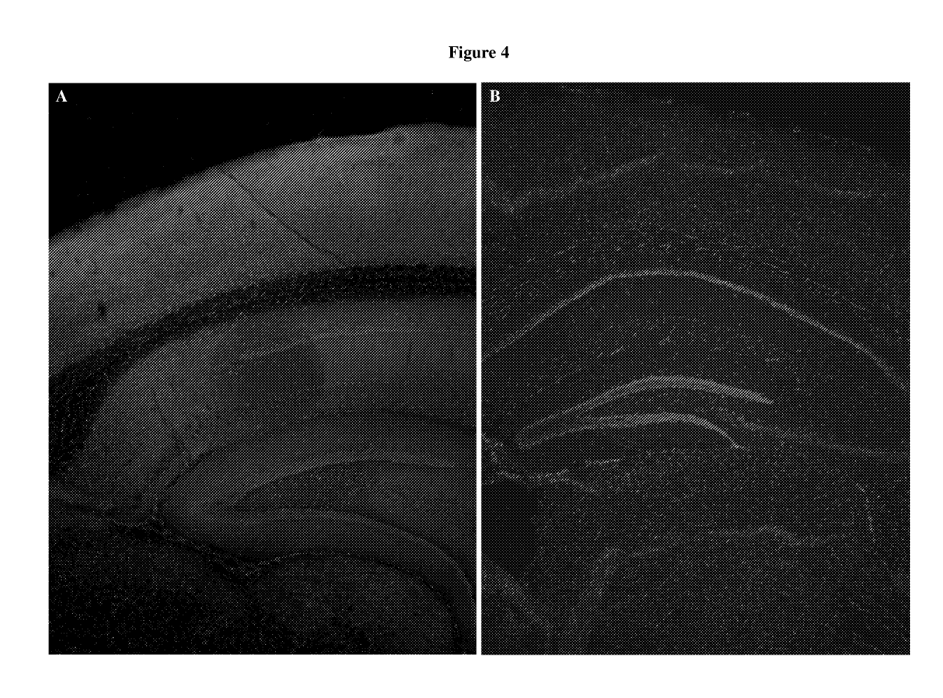

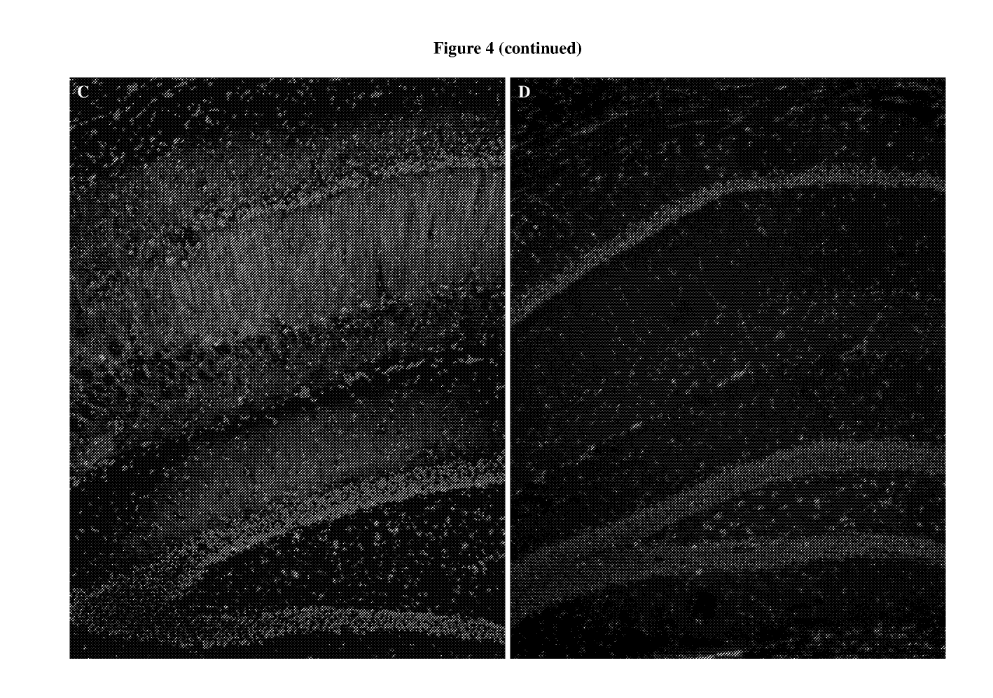

CDK5 substrates were detected in the hippocampus and cortex (FIG. 4, parts A and B). In the hippocampus, a prominent signal was detected in the dendrites of pyramidal neurons of the CA1 region (FIG. 4, parts C and D). The specific signal was detected only in the brain expressing the AS mutant kinase, verifying the kinase-substrate specificity under this condition.

1. A method for in situ visualization of kinase activity in a sample comprising a kinase, the method comprising:
(a) incubating the sample with a fixative;
(b) incubating the sample with an ATP analog, wherein the kinase accepts the ATP analog as a phosphate donor substrate, wherein the γ-phosphate of the ATP analog comprises a transferrable label; and
(c) detecting the transferrable label.

2. The method of claim 1, wherein the fixative comprises an aldehyde.

3. The method of claim 2, wherein the aldehyde is formaldehyde.

4. The method of claim 3, wherein the concentration of formaldehyde is between about 1% and about 10%.

5. The method of claim 4, wherein the concentration of formaldehyde is between about 3% and about 5%.

6. The method of claim 5, wherein the concentration of formaldehyde is about 4%.

7. The method of any one of claims 1-6, wherein the sample is incubated with the fixative for about 10 minutes or shorter.

8. The method of claim 7, wherein the sample is incubated with the fixative for about 5 minutes.

9. The method of claim 1, wherein the fixative comprises an alcohol.

10. The method of any one of the preceding claims, wherein the ATP analog is a derivative of ATP having a substitution group comprising at least three carbon atoms covalently attached to the adenine group of the ATP.

11. The method of claim 10, wherein the substitution group is attached to the N6 position of the ATP.

12. The method of claim 11, wherein the ATP analog is selected from the group consisting of N6-furfuryladenosine-5′-O-(3-thiotriphosphate), N6-(cyclopentyl)ATP, N6-(cyclopentyloxy)ATP, N6-(cyclohexyl)ATP, N6-(cyclohexyloxy)ATP, N6-(benzyloxy)ATP, N6-(pyrolidino)ATP, N6-(ippperidino)ATP, N6-(2-phenylethyl)adenosine-5′-O-(3-thiotriphosphate) and N6-phenyladenosine-5′-O-(3-thiotriphosphate).

13. The method of claim 12, wherein the substitution group is N6-furfuryladenosine-5′-O-(3-thiotriphosphate).

14. The method of any one of claims 1-13, wherein the transferrable label is a thiophosphate.

15. The method of claim 14, wherein detecting the transferrable label comprises alkylating the thiophosphate under suitable conditions to form a thiophosphoester.

16. The method of claim 15, wherein the thiophosphoester comprises a detectable moiety.

17. The method of claim 16, wherein the detectable moiety is selected from the group consisting of a fluorophore, an electron dense moiety, and a moiety specifically binding to a binding protein.

18. The method of claim 17, wherein the detectable moiety is a moiety specifically binding to a binding protein, wherein detecting the transferrable label further comprises incubating the sample with the binding protein.

19. The method of claim 18, wherein the binding protein is an anti-thiophosphoester antibody.

20. The method of claim 18, wherein the detectable moiety is a biotin, and wherein the binding protein is avidin or a homolog thereof.

21. The method of any one of claims 15-20, wherein the suitable conditions comprise an acidic condition.

22. The method of claim 21, wherein the acidic condition has a pH of 6.0 or lower.

23. The method of claim 22, wherein the acidic condition has a pH of 5.0 or lower.

24. The method of claim 23, wherein the acidic condition has a pH of about 4.0.

25. The method of any one of claims 1-13, wherein the transferrable label comprises a first click chemistry handle.

26. The method of claim 25, wherein the first click chemistry handle is an azido group or a propargyl group.

27. The method of claim 25, wherein detecting the transferrable label comprises contacting the first click chemistry handle with a second click chemistry handle.

28. The method of claim 26, wherein the second click chemistry handle comprises a detectable moiety.

29. The method of claim 27, wherein the detectable moiety is selected from the group consisting of a fluorophore, an electron dense moiety, and a moiety specifically binding to a binding protein.

30. The method of any one of claims 1-13, wherein the transferrable label is an uncommon isotope.

31. The method of claim 30, wherein the transferrable label is an uncommon isotope of phosphorus, oxygen or hydrogen.

32. The method of any one of claims 1-30, further comprising a step of quenching a thiol group prior to step (b).

33. The method of any one of claims 1-31, further comprising a step of stopping kinase reaction after step (b).

34. The method of any one of the preceding claims, wherein the kinase is selected from the group consisting of CDC7, AURKA, SRC, TTK, CDK9, CDK12, PLK4, MST3, ALK7, ROCK2, PKD, RET, EGFR, CDK7, ATM, EPHB1, EPHB2, EPHB3, PRKCI, NDR1, AMPKA2, ERK2, JAK1, JAK3, ZAP70, PRKCE, AKT, AURKB, CAMK2, CDK2, CSK, GRK2, JNK2, LCK, MEK1, PKA, PKCD, PLK1, RAFT, SAD1, SYK, TRKA, TRKB, TRKC, and JNK1.

35. The method of claim 33 or claim 34, wherein the kinase comprises at least one amino acid substitution in the kinase domain.

36. The method of claim 33 or claim 34, wherein the kinase comprises an amino acid sequence at least 80% identical to a sequence selected from the group consisting of SEQ ID NOs: 1-12.

37. The method of any one of the preceding claims, wherein the sample comprises a cell.

38. The method of any one of the preceding claims, wherein the sample comprises a tissue or an organ.

39. A kit comprising:
(a) a fixative;
(b) an ATP analog, wherein the γ-phosphate of the ATP analog comprises a transferrable label; and
(c) one or more agents for detecting the transferrable label.

40. The kit of claim 39, wherein the fixative comprises an aldehyde.

41. The kit of claim 40, wherein the aldehyde is formaldehyde.

42. The kit of claim 41, wherein the concentration of formaldehyde is between about 1% and about 10%.

43. The kit of claim 42, wherein the concentration of formaldehyde is between about 3% and about 5%.

44. The kit of claim 43, wherein the concentration of formaldehyde is about 4%.

45. The kit of any one of claims 39-44, further comprising an instruction to incubate a sample with the fixative for about 10 minutes or shorter.

46. The kit of claim 45, wherein the instruction instructs to incubate a sample with the fixative for about 5 minutes.

47. The kit of claim 39, wherein the fixative comprises an alcohol.

48. The kit of any one of claims 39-47, wherein the ATP analog is a derivative of ATP having a substitution group comprising at least three carbon atoms covalently attached to the adenine group of the ATP.

49. The kit of claim 48, wherein the substitution group is attached to the N6 position of the ATP.

50. The kit of claim 49, wherein the substitution group is selected from the group consisting of N6-furfuryladenosine-5′-O-(3-thiotriphosphate), N6-(cyclopentyl)ATP, N6-(cyclopentyloxy)ATP, N6-(cyclohexyl)ATP, N6-(cyclohexyloxy)ATP, N6-(benzyloxy)ATP, N6-(pyrolidino)ATP, N6-(ippperidino)ATP, N6-(2-phenylethyl)adenosine-5′-O-(3-thiotriphosphate) and N6-phenyladenosine-5′-O-(3-thiotriphosphate).

51. The kit of claim 50, wherein the substitution group is N6-furfuryladenosine-5′-O-(3-thiotriphosphate).

52. The kit of any one of claims 39-51, wherein the transferrable label is a thiophosphate.

53. The kit of claim 52, wherein the one or more agents for detecting the transferrable label comprise an agent capable of alkylating the thiophosphate form a thiophosphoester.

54. The kit of claim 53, wherein the thiophosphoester comprises a detectable moiety.

55. The kit of claim 54, wherein the detectable moiety is selected from the group consisting of a fluorophore, an electron dense moiety, and a moiety specifically binding to a binding protein.

56. The kit of claim 54, wherein the detectable moiety is a moiety specifically binding to a binding protein, wherein the one or more agents for detecting the transferrable label further comprise the binding protein.

57. The kit of claim 56, wherein the binding protein is an anti-thiophosphoester antibody.

58. The kit of claim 56, wherein the detectable moiety is a biotin, and wherein the binding protein is avidin or a homolog thereof.

59. The kit of any one of claims 53-58, wherein the one or more agents for detecting the transferrable label further comprise an acidic buffer for the alkylation reaction.

60. The kit of claim 59, wherein the pH of the acidic buffer is 6.0 or lower.

61. The kit of claim 59, wherein the pH of the acidic buffer is 5.0 or lower.

62. The kit of claim 61, wherein the pH of the acidic buffer is about 4.0.

63. The kit of any one of claims 39-51, wherein the transferrable label comprises a first click chemistry handle.

64. The kit of claim 63, wherein the first click chemistry handle is an azido group or a propargyl group.

65. The kit of claim 63 or 64, wherein the one or more agents for detecting the transferrable label comprise a second click chemistry handle.

66. The kit of claim 65, wherein the second click chemistry handle comprises a detectable moiety.

67. The kit of claim 66, wherein the detectable moiety is selected from the group consisting of a fluorophore, an electron dense moiety, and a moiety specifically binding to a binding protein.

68. The kit of any one of claims 39-51, wherein the transferrable label is an uncommon isotope.

69. The kit of claim 68, wherein the transferrable label is an uncommon isotope of phosphorus, oxygen or hydrogen.

70. The kit of any one of claims 39-69, further comprising an agent for quenching a thiol group.

71. The kit of any one of claims 39-69, further comprising an agent for stopping kinase reaction.
